(19) United States
(12) Patent Application Publication
(10) Pub. No.: US 2021/0361236 A1
(43) Pub. Date: Nov. 25, 2021

(54) EARPIECE AND A METHOD FOR DETECTING PHYSIOLOGICAL INFORMATION

(71) Applicant: WELL BEING DIGITAL LIMITED, Pak Shek Kok, New Territories (HK)

(72) Inventor: Ming Yip Wallace WONG, Hong Kong (HK)

(21) Appl. No.: 17/053,083

(22) PCT Filed: May 5, 2019
(86) PCT No.: PCT/CN2019/085438

§ 371 Date: Nov. 5, 2020

(30) Foreign Application Priority Data
May 21, 2018 (HK) 18106626.9

(51) Int. Cl. A61B 5/00; H04R 1/10; A61B 5/026

(52) U.S. Cl. CPC A61B 5/6817; H04R 1/1016; A61B 2562/0233; A61B 2562/185; A61B 5/0261

(57) ABSTRACT

An earpiece comprising a sensor for detecting light in the ear canal. The sensor is held in a cradle to reduce field-of-view. The reduced field-of-view reduce the possibility of light reaching the sensor in a tangential angle. Therefore, the chance of ambient light and light from an emitter reaching the sensor directly is reduced. This improves the likelihood that the readings of the sensor has a greater portion from light that has travelled through the tissue of the ear canal than not.

### FIELD OF INVENTION

The present invention relates to earphones and earpiece devices, having user physiological detection and monitoring capabilities.

### BACKGROUND OF THE INVENTION

Earphones have been provided which include functions for monitoring the pulse of a user wearing the earphones. The earphones may be connected to a portable smartphone or a music playback device. These earphones are installed with emitters and sensors. When the earphone is worn, the emitters and the sensors must be tightly pressed against the ear canal. The emitters emit light in a frequency which can be absorbed by blood in the tissue of the ear canal. The sensors are able to detect the light emitted by the emitters. Some of the light emitted by the emitters penetrates through the skin and tissue of the ear canal and, by being scattered within the ear tissue, is able to emerge from the tissue to be detected by the sensor.

As blood flow in the tissue pulsates by the pumping of the heart, the amount of light which is absorbed by blood increases and decreases according to the pulsating blood content in tissue. Consequently, the amount of light which passes through the tissue to reach the sensors is observed by the sensors as a fluctuation. By signal analysis, the pulse of the user can be observed thereby to deduce his heart condition, blood pressure, fitness and exercise effectiveness, and even psychological stress level.

In many situations, it is desirable that the user wears the earphones for long periods of time, sometimes even all around the clock. This is one of the reasons why the earphone is a good choice for conducting user physiological monitoring, as many modern city dwellers are uses smartphones for a large part of the day. Such long term continual monitoring is particularly important for users who are prone to cardiac arrest, or for users who need to be observed for a long period of time, such as over a few days or weeks, in order to observe change or deterioration in physiological condition.

Unfortunately, the existing earphones having physiological monitoring functions are not suited to be worn for long period of time. In order for the sensors not to pick up ambient light, which adds noise affecting the accuracy of the pulse monitoring, the earplug has to fit tightly against the surface of the ear canal, as explained above. However, a tight fit compresses the tissue of the ear canal. This restricts blood flow. The user soon feels an itch in the ear canal, and may experience pain from wearing the earphone for too long.

Therefore, it is desirable to design an earphone which is able to be worn for an extended period of time more easily than those in the state of the art, to improve the possibility of having an earphone which is useful as a long term physiology monitoring device.

### SUMMARY OF THE INVENTION

In a first aspect, the invention proposes an earpiece comprising: a nozzle for insertion into an ear canal; the nozzle having a cradle; the nozzle installed with a sensor; wherein the sensor is positioned in the cradle.

The cradle limits the field-of-view of the sensor, and this protects the sensor from detecting light which reaches the sensor from a predetermined, undesirable angle. Light from the undeniable angle may possibly be ambient light from the surroundings which travel into the ear canal in an angle that is slightly parallel to the axis of the ear canal, and reaches the sensor in a tangential angle.

By reducing detection of ambient light, the readings of the sensor has less noise components and contains more information on the blood flow in the wearer of the earpiece from light which has travelled through the tissue of the ear canal. In other words, the cradle reduces the baseline of the sensor's detection; the sensor's reading is purer, having thereby an accentuated contribution from light which has travelled though the tissue of the user.

This directly translates into less need to apply signal processing techniques to filter away noise from ambient light or stray noise from any nearby light emitter on the same earphone. If the earpiece contains a processor for processing the readings from the sensor, the processor may now be one which has less processing power, making it cheaper to produce the earphone. A processor having less processing power, or having les processing to do, generates less heat. Hence, if the frequency used to monitor physiological information of the user is in the infrared range, this further reduces noise caused by heat. Reduced likelihood of heat generation makes the earpiece more suited for being worn for longer period of time.

Furthermore, the sensor having a narrower field-of-view means the earpiece does not have to have a dimension which fits tightly into the ear canal of the user in order to avoid reading light that reaches the sensor tangentially. In other words, the narrow field-of-view gives spatial allowance for the earpiece to fit somewhat loosely in the ear canal. This improves the possibility that the earpiece may be worn for extended period of time for reliable, long term physiological monitoring.

Preferably, however, the nozzle comprises a speaker. Hence, an embodiment of the invention includes an earphone, and is not just any earpiece. Alternatively, an embodiment of the invention includes an electronic hearing aid. Many people who require hearing aids are elderly people who might be in need of physiological monitoring by wearing the earpiece.

Typically, the nozzle is provided with at least one emitter; the at least one emitter being positioned in a respective cradle. The cradle limits the field of projection of the emitter. This ensures that light from the emitter may be shone into the tissue of the ear canal, and light which would escape from the emitter to arrive at the sensor in a tangential angle without passing through the tissue of the ear canal is minimised.

By having both the sensor and the emitter in their respective cradles, noise and background interference on the sensor's detection of light containing information of blood flow in the user is reduced.

Preferably, the nozzle is provided with two emitters; each of the two emitters positioned such that light emitted by the two emitters reach the sensor in a cradle from different directions. In this case, the emitters may emit light alternatively to the sensor, since there is only one sensor. Having different travel paths to the sensor allows readings of the sensor to be more accurate, as the light has travelled through different portions of the ear canal tissue, and by averaging the consecutive readings of the sensor of two sources of light. It remains nevertheless advantageous that the sensor is in a cradle since any tangential light from the emitter is unlikely to enter into the cradle to reach the sensor by bouncing off the wall of the ear canal.

However, in yet a more useful embodiment, the light of the emitters is provided at the same time. As tangential light arriving at the sensor is reduced, ambient light causing noise, and light which are merely reflected from the wall of the ear canal, are both reduced. As most of the light which must have passed through the tissue of the ear canal, there is no need to provide light from the emitters to the sensor alternatively. Simply allowing the emitters to emit light at the same time to be detected by the sensor has the same effect as averaging the signals from both emitters in an analogue way. This further reduces need for processing power, which is to switch over between the emitters, and for summing and averaging the readings from the different emitters.

Preferably, a sensor and an emitter are provided as an emitter-and-sensor pair; and the nozzle is provided with a plurality of such emitter-and-sensor pairs.

More preferably, however, each emitter-and-sensor pair is provided with another sensor, such that one emitter and two sensors are provided as a group; and the nozzle is provided with a plurality of such emitter-and-sensors groups; any one of the two sensors in any one of the emitter-and-sensors groups is configured to sense light emitted from an emitter in another one of the emitter-and-sensors groups.

In order for the invention to provide repeatable monitoring of the user's physiological condition, the earphone is preferably designed to be secured to the concha of a user, and material of the part of the earphone to secure into the concha being a hard material. The concha of the ear is not symmetrical, and any device which is shaped to fit into the concha of a user, particularly a tailor-made device, may be repeatedly placed in exactly the same position, time after time.

Preferably, the outer surface of the sleeve is made of a material having the same reflective index the material filling up the sleeve. This reduces the amount of internal reflection which can raise the baseline or create noise to the readings of the sensor.

Preferably, the wall of the cradle is absorbent of the frequency of the light which the sensor is able to detect. Typically, this could be archived by making the wall in a dull, matt black colour, so that most visible wavelengths are absorbed. However, depending on the light emitted by the emitters, the frequency of the light which the sensors is expected to detect, other colours or materials can be used to make up the walls of the cradle. In this way, the walls of the cradle is unlikely to reflect stray light in the same frequency, ensuring most of the light detected by the sensor has penetrated the ear canal wall, and reaches the sensor directly. This increases the purity of the signal as ambient light in the detection frequency arriving at the cradle in a tangential angle is likely to be unable to reach the sensor.

In a further aspect, the invention proposes a method of detecting physiological information from a ear canal, comprising the step of: emitting a light source into the tissue of an ear canal; blocking light which has merely rebounded the wall of the ear canal from reaching a sensor; and the sensor detecting the light from the light source which emerges from the tissue of the ear canal.

Preferably, the step of blocking light which has merely rebounded the wall of the ear canal from reaching a sensor comprises providing a wall, corner or eaves to block the light.

Yet more preferably, the method comprises the further step of absorbing light from the light source using a material which is absorbent of the light where the light is at a pre-determined angle to the sensor.

Typically, the step of absorbing light from the light source using a material which is absorbent of the light where the light is at a pre-determined angle to the sensor comprises providing a material which is absorbent of the light and placing the material in the way of the light which might reach the sensor in the pre-determined angle.

In yet a further aspect, the invention proposes an earpiece comprising a nozzle for insertion into an ear canal; the nozzle has a triangular cross-section along the axis for insertion into the ear canal; the nozzle installed with a sensor on one side of the nozzle, the side being according to a side of the cross-sectional triangle.

In this case, there is no need for a cradle which holds any sensor snugly. The corner of the triangle shaped nozzle is able to provide a small measure of blockage, preventing a certain amount of light from arriving at the sensor tangentially. While this may not be as efficient as using a cradle, any corner, eaves or blockage which prevents some light from reaching the sensor tangentially will improve the purity of the light reaching the sensor by passing through ear canal tissue.

Typically, the nozzle is also provided with at least one emitter; the at least one emitter positioned on a different side of the nozzle, the side being according to another side of the cross-sectional triangle.

### BRIEF DESCRIPTION OF THE FIGURES

It will be convenient to further describe the present invention with respect to the accompanying drawings that illustrate possible arrangements of the invention, in which like integers refer to like parts. Other arrangements of the invention are possible, and consequently the particularity of the accompanying drawings is not to be understood as superseding the generality of the preceding description of the invention.

FIG. 1 is an illustration of an embodiment;

FIG. 2A is a cross sectional view of a part of the embodiment of FIG. 1;

FIG. 2B is a cross sectional view of a part of the embodiment of FIG. 1;

FIG. 2C is a variation of the embodiment shown in cross sectional view in FIG. 2A;

FIG. 2D is a perspective view of the drawing of FIG. 2A;

FIG. 3 shows the embodiment of FIG. 2 in use;

FIG. 4 shows the embodiment of FIG. 2 in use;

FIG. 5 is a comparative example to the embodiment shown in cross sectional view in FIG. 2A;

FIG. 6 is another variation of the embodiment shown in cross sectional view in FIG. 2A;

FIG. 7 shows the embodiment of FIG. 6 in use;

FIG. 8 is a comparative example to the embodiment shown in cross sectional view in FIG. 7;

FIG. 9 is another variation of the embodiment shown in cross sectional view in FIG. 2A;

FIG. 9A is a perspective view of the embodiment of FIG. A;

FIG. 10 is a comparative example to the embodiment shown in cross sectional view in FIG. 9;

FIG. 11 illustrates a human ear;

FIG. 12 illustrates how an embodiment of the invention fits into a human ear;

FIG. 13 shows a step is the making of the embodiment of FIG. 12;

FIG. 14 shows a further step is the making of the embodiment of FIG. 12;

FIG. 15 shows yet a further step is the making of the embodiment of FIG. 12;

FIG. 16 shows the step of FIG. 15 completed;

FIG. 17 shows yet another variation of the embodiment shown in FIG. 6

FIG. 18 is a cross sectional view of a part of a variation of the embodiment of FIG. 1,

FIG. 19 is a perspective of the embodiment of FIG. 18; and

FIG. 20 is a variation of the embodiment of FIG. 11.

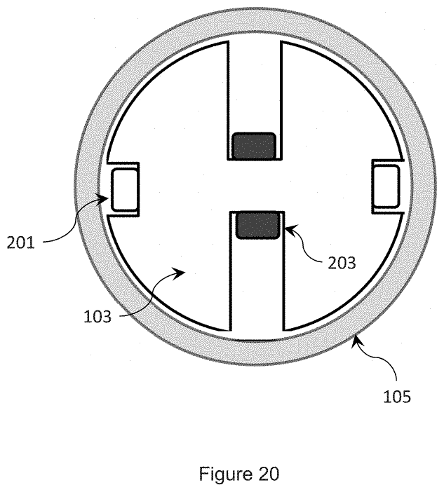

### DETAILED DESCRIPTION OF EMBODIMENTS

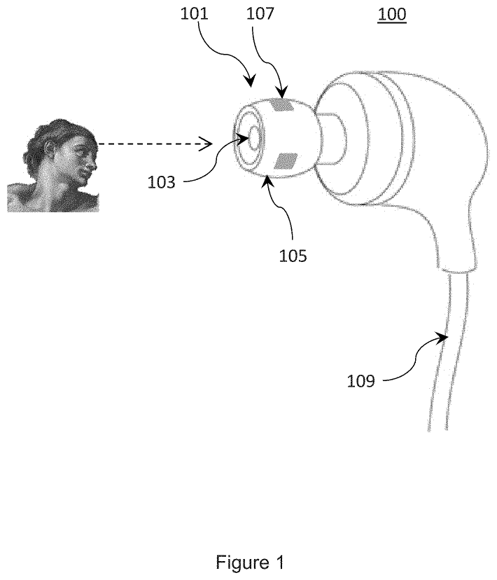

FIG. 1 is an earphone 100. The earphone comprises a plug. The plug is the part of the earphone which is meant to be inserted into the ear canal of the user. The earphone has an arrangement 107 of sensors and emitters on the plug 101. The plug extends from a housing. A wire extends from the housing to be connected to the output port of a smartphone. Circuitry may be provided in the housing which includes a processor for processing information obtained by the sensors, and for controlling the operation of the sensors and the emitters.

The plug is made of several parts, including a nozzle 103 and a sleeve 105. The nozzle is positioned in the centre of the sleeve, and is usually an elongate piece such as a cylinder. The image of a man in the drawing is into the axis of the nozzle. Within the nozzle is fitted a diaphragm (not illustrated) for producing sound that may be heard by a user wearing the earphone. The nozzle is capped with the sleeve in order to protect the nozzle. The sleeve is intended to be inserted into the user's ear canal.

The nozzle is also provided with light emitters and light sensors. The emitter emits light in a frequency which can be absorbed by blood in the tissue forming the ear canal. Light from the emitted passes through the sleeve into the tissues of the ear canal. Hence, the sleeve is typically made of a translucent or transparent material, which may be plastic such as acrylic or glass, and which allows light from the emitter to pass through to reach the ear canal wall, and to allow light scattering from the ear canal tissue to pass through to reach the sensor.

Some of the light from the emitter which has penetrated into the tissue of the ear canal is, by being scattered within the tissue, able to emerge from the tissue into the ear canal, and passes through the sleeve to be detected by the sensor.

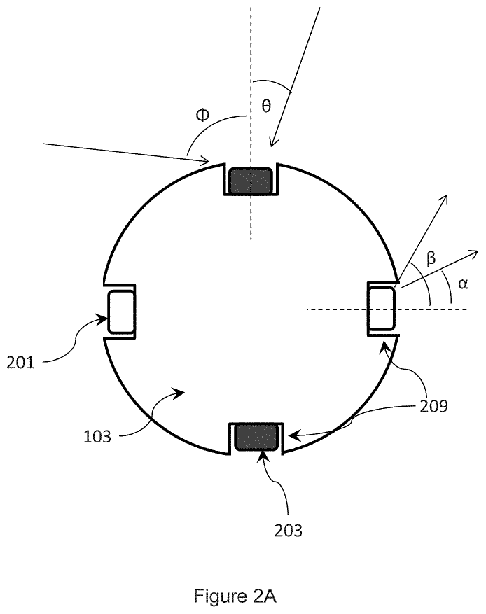

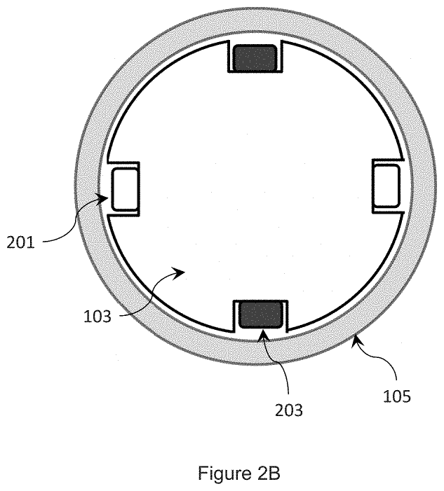

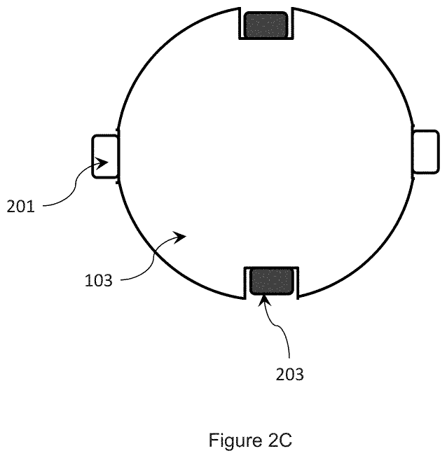

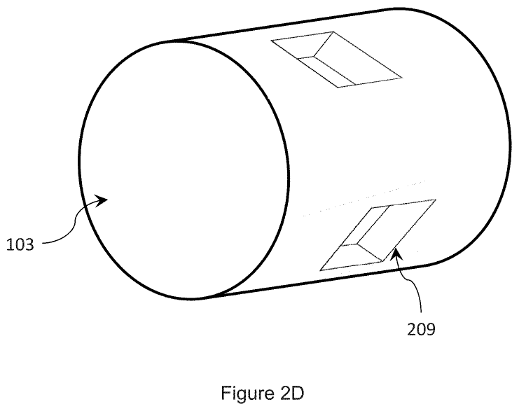

FIG. 2A illustrates a schematic diagram of the cross-section of the nozzle having two emitters 201 and two sensors 203. The cross-section is in the view of the man illustrated in FIG. 1. The black rectangles in FIG. 2A represent sensors while the white rectangles represent emitters. The sensors and emitters are arranged generally around the circumference of the nozzle. Each sensor is disposed between the two emitters and each emitter is disposed between the two sensors.

The sensor is situated in a cradle 209 in the nozzle. In this embodiment the cradle is a concave depression carved into the surface of the nozzle. A drawing of the nozzle showing concave depressions, being the cradles, in the surface is provided as FIG. 2D.

The cradle reduces the field-of-view of the sensor. Hence, light from a tangential angle relative to the position of the sensor on the nozzle is restricted from reaching the sensor. The narrow field-of-view provides that most of the light which the sensor detects reaches the sensor in an angle generally acute to the normal of the sensor, i.e. substantially more light is likely to reach the sensor about the angle θ then about the angle ϕ.

FIG. 2B is similar to FIG. 2A except that the sleeve is now included in the illustration, shown covering over the nozzle.

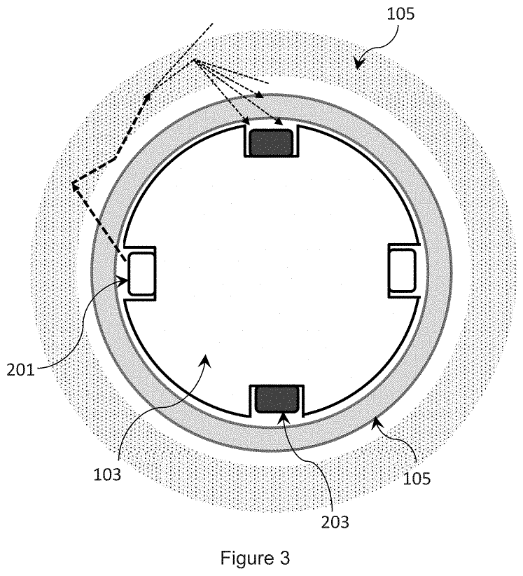

FIG. 3 illustrates how light from one of the emitters is able to reach one of the sensors. Light, show in dashed lines, emitted from the emitter penetrates the skin of the ear canal and travels within the tissue. The light is naturally scattered within the tissue. Some of the light is absorbed by blood in the tissue and is converted into heat or other form of energy. Some of the light is re-emitted in the same frequency, and some is simple reflected internally within the tissue. As a result, the light is simply scattered. Some of the scattered light exits the tissue into the ear canal and reaches the sensor. The light reaching the sensor has a pulsating intensity which caused by the pulsating volume of blood in the tissue. In this way, the pulse of the user may be observed.

In FIG. 2A, the emitters are shown also placed in cradles although this may not be necessary in some embodiments. For example, as illustrated in FIG. 2C, a variation of the embodiment of FIG. 2A may have emitters which are placed on the nozzle but not within any cradle. The sensors, however, remain protected from some light which could reach the sensors tangentially.

The embodiment of FIG. 2C may be relatively less effective than the embodiment of FIG. 2A in preventing tangential light from reaching the sensors. This is because, by placing the emitters in cradles as shown in FIG. 2A, light which emits generally sidewise from the emitters are already blocked by the walls of the cradle. Hence, most of the light leaves the cradle in a generally acute angle to the normal of the positon of the emitter, i.e. substantially more light is likely to leave the emitter about the angle α than about the angle β. Consequently, the amount of light reaching the wall of the ear canal in an acute angle to the illustrated normal is more than the amount of light reaching the wall of the ear canal in an angle less acute to the illustrated normal. This reduces the amount of light which has not passed through the tissue of the ear canal but has merely bounced off the wall of the ear canal to reach the sensor. As a result, the amount of light that carries information on the pulsation of blood in the ear canal is relatively higher than the amount of light that carries no such information. This increases the efficiency of the embodiment in detecting the pulses of the user, and reduces noise signals. In contrast, the embodiment of FIG. 2C may still benefit from the sensors being held in cradles, as the cradles themselves are able to block light reaching the sensors tangentially. However, the emitters are not held in cradles, there is a greater chance of light simply bouncing off the wall of the ear canal, and then bouncing off the surface of the sleeve, and finally reaching the sensors after bouncing off the surfaces multiples times without having entered into the ear canal tissue.

As the skilled man knows, the more tangential the angle of incidence to a surface, the greater the likelihood that a ray of light may rebound by reflection from the surface instead of penetrating into the surface.

Generally, the deeper the cradle which is containing the sensor, the more likely it is that light has to reach the sensor in an angle which is generally acute to the normal of the sensor's position. Hence, the light reaching the sensor is more likely to have passed through the tissue of the ear canal.

Similarly, the deeper the cradle which is containing the emitter, the more the light leaving the emitter has to be in an angle which is generally acute to the normal of the emitter's position. Hence, the light reaching the sensor is less likely to have not passed through the tissue of the ear canal.

Furthermore, the skilled man would appreciate that the wider the mouth of cradle containing the sensor, the greater the chance that more light might reach the sensor in a tangential angle. Similarly, the wider the mouth of the cradle containing the emitter, the greater the chance that more light might leave the emitter in a tangential angle, despite the depth of the cradle. However, the optimal depth and width of the cradle in an actual product varies according to the size of the earphone, the size of the nozzle, the size of the emitters and the sensors, as well as the intensity of light from the emitters. This is a matter of the product specification, and does not require elaboration here.

Preferably, the nozzle is made of a material that is not reflective of the light which is emitted by the emitter. A black-coloured nozzle is useful for most embodiments in which the emitter light is within the visible frequency range, which helps to absorb light which reaches the surface of the nozzle, preventing reflection off the surface of the nozzle. This ensures better darkness in the ear canal light, such that light that the sensors detect is more likely than not to have passed through the ear canal tissue. Accordingly, the light reaching the sensor is like to be information-rich relating to the blood content and blood pulsation in the tissue of the ear canal

In a the preferred one of the simplest embodiments, the cradle for the sensor is made as deep as possible, like a pin-hole, and the walls of the cradle is black or, depending on the light used, made to be as absorbent of light in that frequency as much as possible. In this way, light reaching the sensor is more like light emanating from travelling through the ear canal flesh instead of light which has bounced off the canal wall and also bounced off the cradle wall, preserving purity of information.

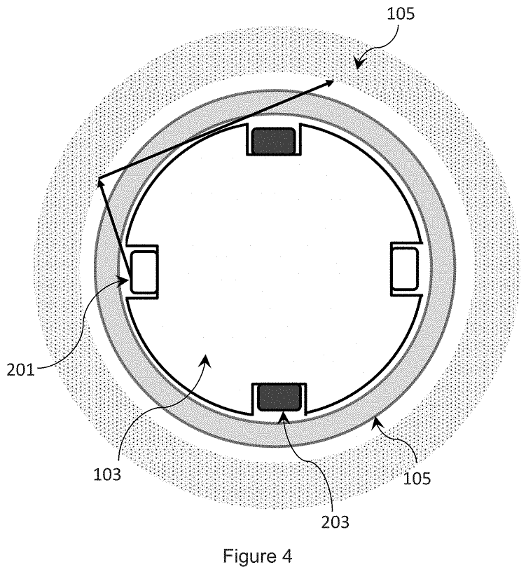

FIG. 4 illustrates how light, shown in solid line, from the emitter is simply reflected by the surface of the skin and merely rebound to the sensor without first penetrating into the tissue. This light signal is unable to provide any information relating to the fluctuation of blood content in the tissue and not useful for the purpose of monitoring the physiological information of the user.

Advantageously, the narrow field-of-view gives spatial allowance for the earphone to fit somewhat loosely in the ear canal, i.e. the sensor does not have to be pressed tightly against the wall of the ear canal to prevent tangential light from reaching the sensor. This improves the possibility that the earphone may be worn for extended period of time for reliable, long term physiological monitoring.

Typically, in embodiments which comprise two or more emitters, the emitters emit light alternatively, one after another, so that each sensor is detecting light emitted from emitter after the other. However, in yet a more useful embodiment, all the emitters emit light at the same time. Information on the pulsation of blood in the tissue will not be undermined by the flooding of light by the emitters, as tangential light arriving at the sensor is prevented by the cradles. That is, ambient light from outside the ear causing noise, and light emitted by the emitters which are merely reflected from the wall of the ear canal, are both prevented. Accordingly, as most of the light must have passed through the tissue of the ear canal, there is reduced need to emit light from the emitters alternatively. Simply allowing the emitters to emit light at the same time to be detected by the sensor has the same effect as summing and averaging the signals from both emitters in an analogue way. This also reduces need for processing power to switch over between the emitters, and for averaging the readings from the different emitters.

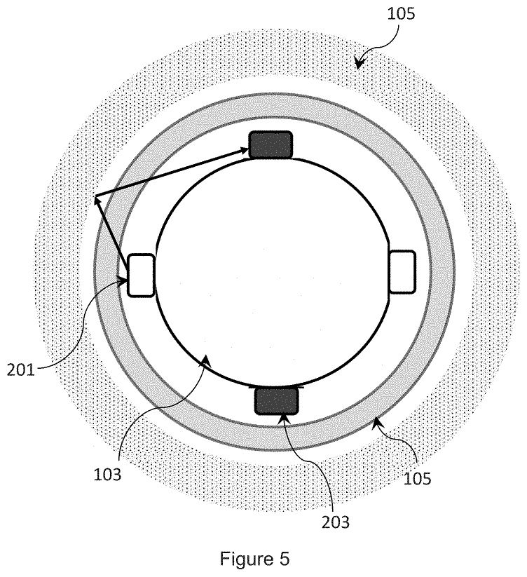

FIG. 5 is a comparative example, showing a prior art device. The sensors are installed into the surface of the nozzle. Hence, the sensor is exposed to light which may reach the sensor tangentially, shown in solid lines, passing through the transparent or translucent sleeve over the nozzle and rebound from the surface of the ear canal without having entered into the flesh of the ear canal. This causes noise in the reading of the blood pulsation by the sensor. Therefore, the observing of the pulse of the user is not accurate and requires more treatment by signal processing techniques.

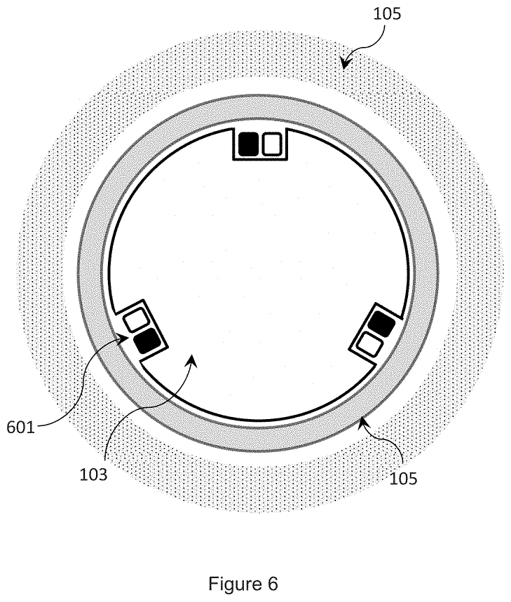

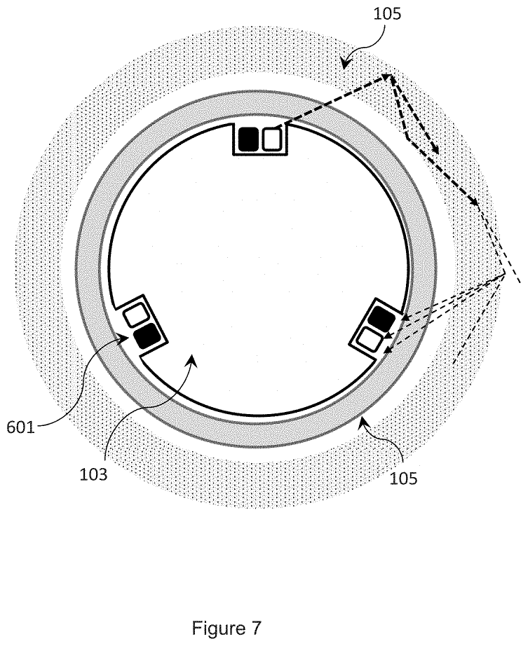

FIG. 6 shows another embodiment of the present invention in which the sensors and emitters are provided in sensor and emitter pairs. Three sensor-emitter pairs are shown place around a nozzle having a circular cross section. FIG. 7 shows how the emitter of one emitter-and-sensor pair emits light, shown as dashed lines, which travels though the tissue of an ear canal and reaches the sensor of an adjacent emitter-and-sensor pair. Again, as in one of the earlier described embodiments, the emitters emit light alternatively, one after another. The sensor in the emitter-and-sensor pair does not operate to sense light when the paired, operating emitter is emitting, such that only the sensors in the adjacent pairs are able to sensing light from the operating emitter. In other words, each one of the emitter-and-sensor pairs takes turn to emit light while the other two pairs sense emitted light.

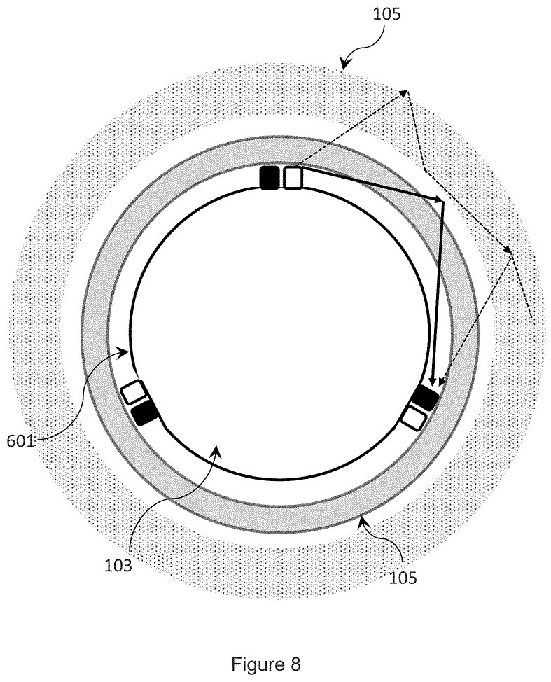

FIG. 8 is another comparative example showing how some of the light, illustrated by the solid lines, from an emitter may simply reflect off the wall of the ear canal without penetrating into the tissue of the ear canal, and arriving at the sensor of the adjacent emitter-and-sensor pair. The emitter-and-sensor pairs are not held in any cradles. Therefore, this allows the sensor to pick up the tangential light, which has a high proportion of light rebounding directly from the wall of the ear canal without having entered into the ear canal tissue. For a more completion depiction of the reality, FIG. 8 also shows some light from the emitter which has penetrated the skin of the ear canal and into the tissue, shown in dashed lines, reaching the adjacent sensor. However, this amount of light that has penetrated the skin of the ear canal is mixed with light which has not, causing aberration of physiological signal and data.

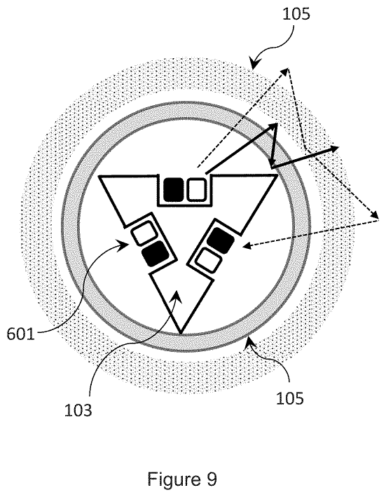

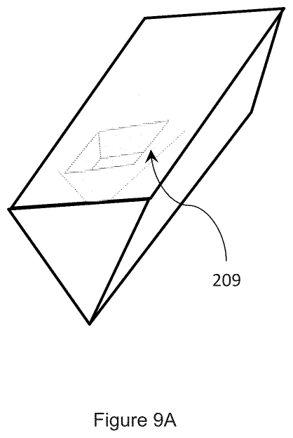

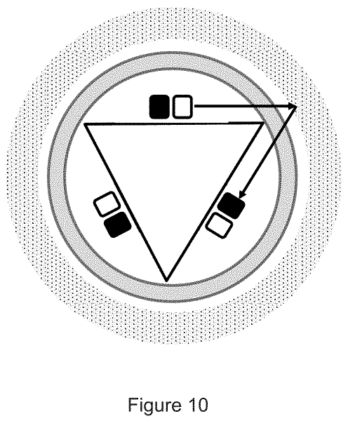

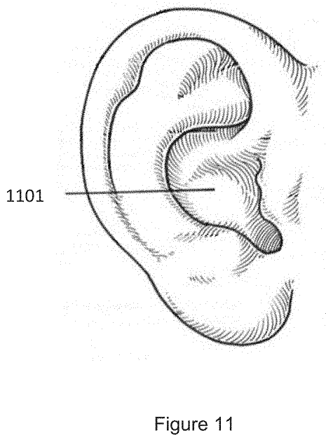

FIG. 9 illustrates another example in which the nozzle has a triangular cross section. Along with the cradles in which the emitter-and-sensor pairs are held, the edges of the triangle help to block light, shown in solid lines, from reaching the cradled sensors directly from the adjacent emitter. At the same time, a portion of light, shown in dashed line, emitted from the emitter reaches the cradled sensor by passing through the tissue of the ear canal. FIG. 9A is perspective view of the nozzle of FIG. 9, showing a concave depression, being a cradle 209, in the surface of the nozzle. FIG. 10 is a comparative example of what happens if the emitter-and-sensor pairs of FIG. 9 are not shielded by being placed within a cradle. As illustrated by the arrows in solid line representing a light ray from an emitter, the ray can simply bounce off the wall of the ear canal to arrive at the sensor in the adjacent emitter-and-sensor pair. FIG. 11 is an illustration of the human ear. The part of the ear called the concha 1101 is used nowadays to place earphones which are custom made.

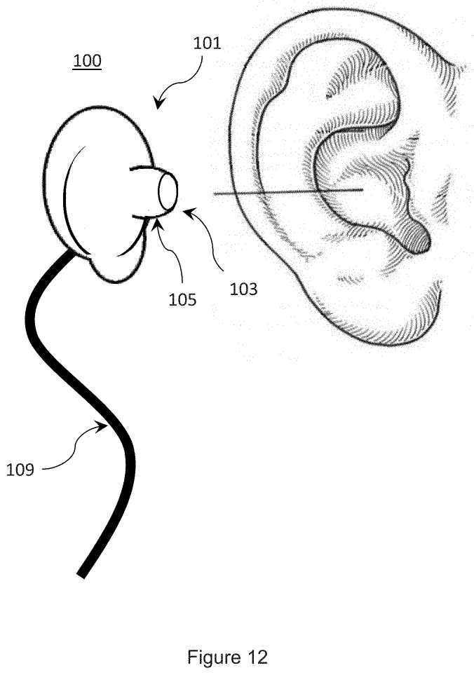

FIG. 12 shows a further improvement of the embodiments in which the earphone is tailor-made to the shape of the user's ear. Typically, such an earphone comprises a part which is moulded to the shape of the concha of the ear. This part is intended to be held by the concha. Being moulded to fit the concha means there is no need for a deformable material to be used to be compressed into the ear in order to provide an interference fit to the ear. An interference fit is a fastening between two parts achieved by friction after the parts are pushed together.

As shown in the drawing, the sleeve part of the earphone is meant to be inserted into the ear canal, as explained also for the earlier described embodiments. The sleeve is also made of the same material as the rest of the earphone meant to be held in the concha. The material is a transparent and hard plastic material such as acrylic, or is made of glass. To make such an earphone, the ear concha of a specific user is first filled with a material which can be cured and hardened over a short period of time. When the material has hardened, the material would have taken on the shape of the concha and the ear canal. The material is then removed from the ear and is used as a mould block to cast a mould in the shape of the concha and the ear canal.

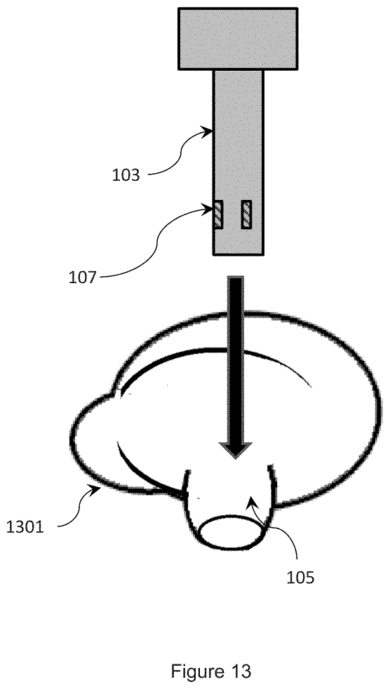

The mould is then used to cast a hollow housing 1301, which is shown in FIG. 13, which has the outer shape of the concha. The housing has a sleeve 105 which can fit into the ear canal. The sleeve is cast to be hollow and is capable of being inserted with the nozzle.

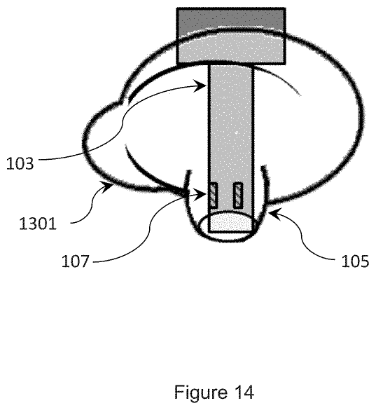

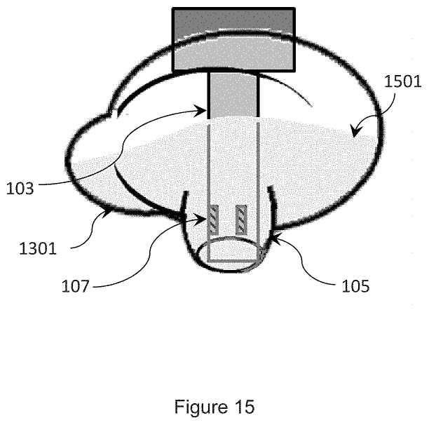

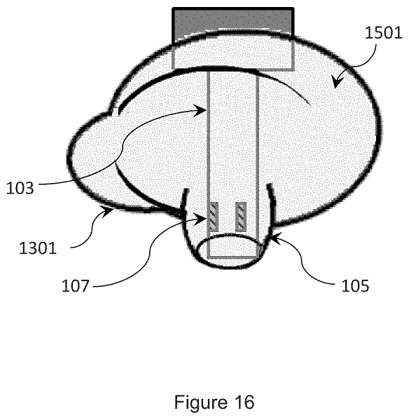

FIG. 14 shows the nozzle positioned into the sleeve part of the hollow of the sleeve. After the nozzle is inserted into the hollow housing, as illustrated in FIG. 15 and then FIG. 16, a material 1501 which is the same as the material used to cast the housing is used to fill up the housing, and to encapsulate the nozzle. When the material has hardened such that the nozzle is firmly held in place within the sleeve, the earphone is made.

Using the same material as used for the sleeve to fill up the hollow prevents internal reflection from the edge of the house, allowing more light to pass from the emitter on the nozzle, out of the housing and to the ear canal. This improves the sensor's ability to detect the pulse of the user in two ways. Firstly, the amount of light leaving the earbud is greater with minimal internal reflection within the housing. Secondly, the amount of light entering into the sleeve from the ear canal is also greater, as there is also reduced chance of light being reflected from the surface of the housing. In contrast, if two different materials are used, one for the outer shape of the sleeve and the other to fill up the sleeve, some light may be reflected away at where the sleeve material and the material filling the sleeve contact. The chance of light being reflected away depends on the difference in refractive index between the two materials. This is known science and elaboration does not require elaboration here.

The hard material used to make the earphone and the tailored fit of the earphone to the concha provide the possibility of positioning the earphone in the same location in ear, virtually every time. The concha of the ear is not symmetrical, and any device which is shaped to fit into an asymmetrical shape is unable to rotate from the intended position in the concha. The repeatability of position allows long term observation of the user's pulse signals even if the user were to take the earphone off occasionally. In other words, the embodiment provides the possibility that it does not matter if there is any part of the tissue in the ear canal that has different blood vessel densities, which could have affected meaningful concatenation of pulse information if the sensors were not placed in the same position every time the pulse is monitored.

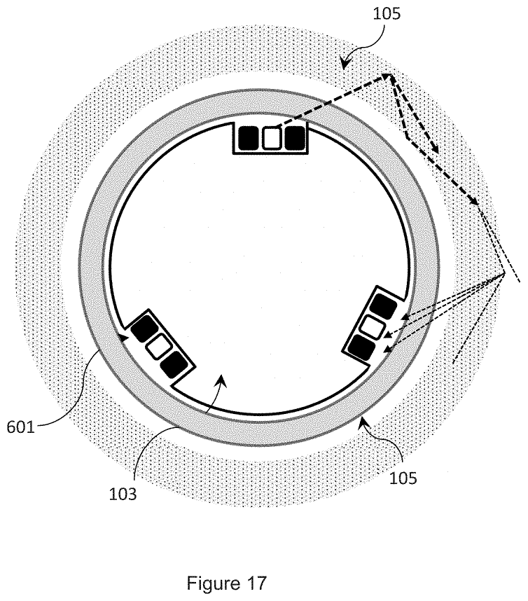

FIG. 17 shows yet another variation of the embodiment shown in FIG. 6, in which the sensors are provided as groups having two or more sensors. That is, where a position on the nozzle is supposed to have an emitter-and-sensor pair, there is now a further sensor, causing the emitter-and-sensor pair to become a group of two sensors and one emitter. When the material is used to fill up the sleeve as shown in FIG. 16, bubbles may be formed permanently within the hardened material. The likelihood of bubbles forming depends on chance and, if this happens, the bubble may be in the way of light reaching from the ear canal wall to a sensor on or in the nozzle in the earphone. To mitigate this problem, the additional sensor can be used instead of the other one in the group if it is detected that the other sensor is suffering from low reading sensitivity.

The material of choice depends on what's available in the industry and a choice may be made studying the reflective index, transparency and durability of the material suitable for the functioning of the emitters and sensors. This is a matter of design and product specification and does not require elaboration here.

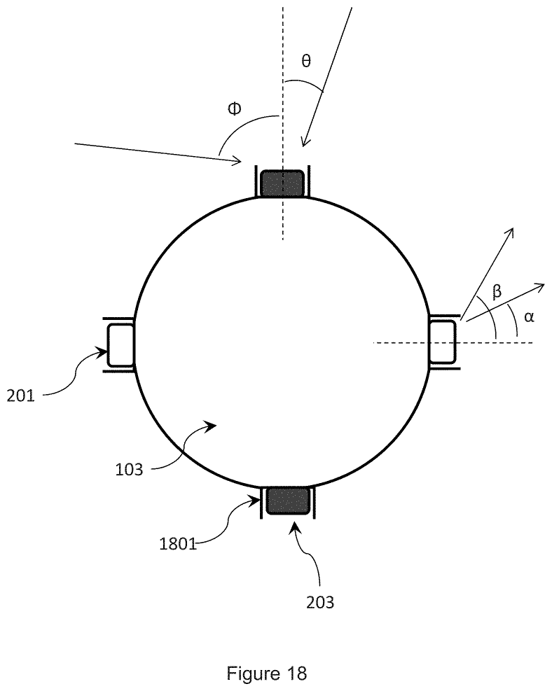

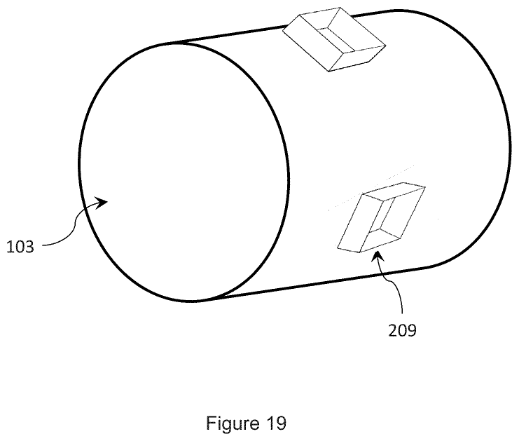

Although the cradle shown in the drawings are depressions provided on the nozzle, it is also possible that the cradles are hollow protrusions on the nozzle. This is illustrated in FIG. 18, which is a variation of the embodiment of FIG. 1. A perspective view is provided as FIG. 19. More specifically, the cradles are provided by a raised continuous lip 1801 on the surface of the nozzle. The lip provides the function of shielding the sensors from light which could reach the sensors in a tangential angle, i.e. substantially more light is likely to reach the sensor about the angle θ then about the angle ϕ. Similarly, the raised lip prevents light from leaving the cradled emitter in a tangential angle, i.e. substantially more light is likely to leave the emitter about the angle α than about the angle β.

Furthermore, it is envisaged that some cruder embodiments of the invention may be simply like the embodiment as shown in FIG. 10. Although FIG. 10 is mentioned as a comparative example, it may still be within the contemplation of the invention. That is, despite not having a cradle to place the sensor or the emitter into, the corners of the triangular nozzle provides some of the functions of a cradle, which is a corner or eaves to block tangential light. The design of FIG. 10 may not be as effective as having a cradle carved into the nozzle but it does goes certain way to improve over the prior art of FIG. 8, for example.

While there has been described in the foregoing description preferred embodiments of the present invention, it will be understood by those skilled in the technology concerned that many variations or modifications in details of design, construction or operation may be made without departing from the scope of the present invention as claimed.

For example, although embodiments have been described as an earphone, i.e. the nozzle comprises a speaker, in an alternative embodiment, the invention includes a hearing aid, or an earpiece without any other function except for physiological data monitoring.

Furthermore, although a wire extends from the housing to be connected to the output port of a smartphone has been described, the skilled reader understands that a wireless earphone which communicates with a smartphone or other devices is included in the invention, such as those which communicate using Bluetooth or Wi-Fi.

Furthermore, although the described embodiments comprise two or more emitters, the skilled reader would appreciate that embodiments which comprise only one emitter are within the contemplation of the invention.

Furthermore, it is possible that the nozzle of the earphone does not have any speaker function, and only has the emitters and sensors, in which case the embodiment is just an earpiece.

Furthermore, some embodiments do not have any emitters installed onto the nozzle. In this case, the source of light that travels through the tissue and exit the wall of the ear canal may be provided by a separate device which may be installed in a part of the body near the ear canal, and which provides light emission shining through the ear canal tissue to arrive at the sensor.

Therefore, in the simplest form, the embodiments comprise an earpiece comprising: a nozzle for insertion into an ear canal; the nozzle having a cradle; the nozzle installed with a sensor; wherein the sensor is positioned in the cradle. In most embodiments, however, the nozzle is provided with at least one emitter, each of the emitter is positioned in a cradle.

1. An earpiece comprising:
a nozzle for insertion into an ear canal;
the nozzle having a cradle;
the nozzle installed with a sensor; wherein
the sensor is positioned in the cradle.

2. An earpiece as claimed claim 1, wherein:
the nozzle comprises a speaker.

3. An earpiece as claimed claim 1, wherein:
the nozzle is provided with at least one emitter;
the at least one emitter positioned in the cradle.

4. An earpiece as claimed claim 1, wherein:
the nozzle is provided with two emitters;
each of the two emitters positioned such that
light emitted by the two emitters reach the sensor in a cradle from different directions.

5. An earpiece as claimed claim 1 wherein:
a sensor and an emitter are provided as an emitter-and-sensor pair, the emitter-and-sensor pair being held in the same cradle; and
the nozzle is provided with a plurality of such emitter-and-sensor pairs held in respective cradles;
the sensor in any one of the emitter-and-sensor pairs is configured to sense light emitted from an emitter in another one of the emitter-and-sensor pairs.

6. An earpiece as claimed claim 5 wherein:
the emitter-and-sensor pair is provided with another sensor, such that one emitter and two sensors are provided as a group; and the nozzle is provided with a plurality of such emitter-and-sensors groups held in respective cradles;
any one of the two sensors in any one of the emitter-and-sensors groups is configured to sense light emitted from an emitter in another one of the emitter-and-sensors groups.

7. An earpiece as claimed claim 5, wherein:
the outer surface of the sleeve is made of a material having the same reflective index the material filling up the sleeve.

8. An earpiece as claimed in claim 1, wherein:
the earphone is designed to be secured to the concha of a user, and material of the part of the earphone to secure into the concha being a hard material.

9. An earpiece as claimed claim 1, wherein:
the wall of the cradle is absorbent of the frequency of the light which the sensor is able to detect.

10. A method of detecting physiological information from an ear canal, comprising the step of:
emitting a light source into the tissue of an ear canal;
blocking light which has rebounded from the wall of the ear canal from reaching a sensor; and
the sensor detecting the light from the light source which emerges from the tissue of the ear canal.

11. A method of detecting physiological information from an ear canal, as claimed claim 10, wherein
the step of blocking light which has rebounded from the wall of the ear canal from reaching a sensor comprises providing a wall, corner or eaves to block the light.

12. A method of detecting physiological information from an ear canal, as claimed claim 10, comprising the further step of:
absorbing light from the light source using a material which is absorbent of the light where the light is at a pre-determined angle to the sensor.

13. A method of detecting physiological information from an ear canal, as claimed claim 10, wherein the step of absorbing light from the light source using a material which is absorbent of the light where the light is at a pre-determined angle to the sensor comprises
providing a material which is absorbent of the light and placing the material in the way of the light.

14. An earpiece comprising:
a nozzle for insertion into an ear canal;
the nozzle has a triangular cross-section along the axis for insertion into the ear canal;
the nozzle installed with a sensor on one side of the nozzle, the side being according to a side of the cross-sectional triangle.

15. An earpiece as claimed claim 14, wherein:
the nozzle is provided with at least one emitter;
the at least one emitter positioned on a different side of the nozzle, the side being according to another side of the cross-sectional triangle.

16.-17. (canceled)
